(19) United States
(12) Patent Application Publication
(10) Pub. No.: US 2009/0320836 A1
(43) Pub. Date: Dec. 31, 2009

(54) Method For Regulating Treatment Based On A Medical Device Under Closed-Loop Physiologic Control

(76) Inventor: Clark R. Baker, JR., Newman, CA (US)

Correspondence Address: NELLCOR PURITAN BENNETT LLC;ATTN: IP LEGAL, 6135 Gunbarrel Avenue, Boulder, CO, 80301, US

(21) Appl. No.: 12/165,230

(22) Filed: Jun. 30, 2008

(51) Int. Cl. A61M 16/10

(52) U.S. Cl. 128/203.14

(57) ABSTRACT

Embodiments of the present disclosure are directed to a system and method for automated medical treatment. Specifically, the disclosure is directed to a first controller capable of monitoring a physiological parameter of a patient and supplying the patient with a delivery parameter based at least in part on the physiological parameter to control the physiological parameter. Further, present embodiments include a second controller capable of determining a therapeutic procedure to be performed on the patient based at least in part on analysis of the delivery parameter, and automatically initiating an alert indicative of the therapeutic procedure and/or automatically performing the therapeutic procedure.

### BACKGROUND OF THE INVENTION

1. Field of the Invention

The present disclosure relates generally to a method and system for controlling a physiological parameter, medical treatment, and/or symptom alert mechanisms with multiple controllers.

2. Description of the Related Art

This section is intended to introduce the reader to various aspects of art that may be related to various aspects of present embodiments, which are described and/or claimed below. This discussion is believed to be helpful in providing the reader with background information to facilitate a better understanding of the various aspects of present embodiments. Accordingly, it should be understood that these statements are to be read in this light, and not as admissions of prior art.

Certain patient physiologic conditions may be achieved or maintained using medical devices designed for such purposes. For example, mechanical ventilators may be utilized to maintain a level of oxygenation in a patient's arterial blood or to achieve a desired arterial pH level. These medical devices may be manually or automatically operated and adjusted based on a comparison of measured and desired physiologic parameter values. For example, upon sensing a blood oxygenation level below a desired value in a patient, an operator could manually adjust a ventilator to provide additional oxygen to the patient. In another example, based on detection of blood oxygenation above or below a desired level in a patient, a device may utilize closed-loop control to respectively reduce or increase the oxygen provided to the patient.

Automated control of patient physiologic parameters may be achieved using various types of control devices and/or algorithms to operate medical devices. For example, computer-based controllers, such as proportional (P) controllers, proportional-integral (PI) controllers, proportional-derivative (PD) controllers, or proportional-integral-derivative (PID) controllers may be utilized to control the output of a medical device based on a measured physiologic parameter value. Use of such automated controllers to operate medical devices based on measured physiological parameters may be more efficient and accurate than manual operation.

### BRIEF DESCRIPTION OF THE DRAWINGS

Advantages of embodiments of the present disclosure may become apparent upon reading the following detailed description and upon reference to the drawings in which:

FIG. 1 is a block diagram of a medical system including a first control feature that induces, maintains, or controls a physiological parameter of a patient, and a second control feature that utilizes output generated from the first control feature to regulate a separate medical therapy and/or facilitate identification of a condition for alarm in accordance with an embodiment of the present disclosure;

FIG. 2 is a block diagram of a medical system including a first control feature that induces, maintains, or controls a patient's blood oxygen saturation, and a second control feature that utilizes control aspects from the first control feature to control a separate therapeutic device and/or notification mechanism in accordance with an embodiment of the present disclosure; and

FIG. 3 is a block diagram of a method illustrating an embodiment of the present disclosure.

### DETAILED DESCRIPTION OF SPECIFIC EMBODIMENTS

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions may be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Embodiments of the present disclosure are directed to using the output of a medical device tinder closed-loop physiologic control to regulate a separate medical therapy, device, or treatment. Specifically, for example, present embodiments are directed to automated control of medical devices to regulate (e.g., maintain, increase, or decrease) at least one physiologic parameter (e.g., a blood oxygenation level, a blood or tissue carbon dioxide level, a heart rate, a blood pressure level, a respiration rate, and/or a tissue oxygenation level) of a patient, and automated control of a separate medical therapy and/or alert mechanism based at least in part on an output resulting from control of the at least one physiologic parameter.

Exemplary embodiments of the present disclosure may control various different physiologic parameters using various different delivery parameters. For example, oxygen delivery may be adjusted based on a patient's estimated blood oxygen saturation level (SpO₂), a ventilator rate may be adjusted based on end tidal carbon dioxide (EtCO₂), continuous positive airway pressure (CPAP) may be adjusted based on SpO₂, and/or patient-controlled analgesia may be inhibited during periods of low SpO₂. Control of a patient's blood oxygen content with oxygen delivery is specifically discussed below as a particular example of present embodiments. However, one of ordinary skill in the art will recognize that this is merely one example and that different control parameters, delivery parameters, and the like may be utilized in accordance with present embodiments.

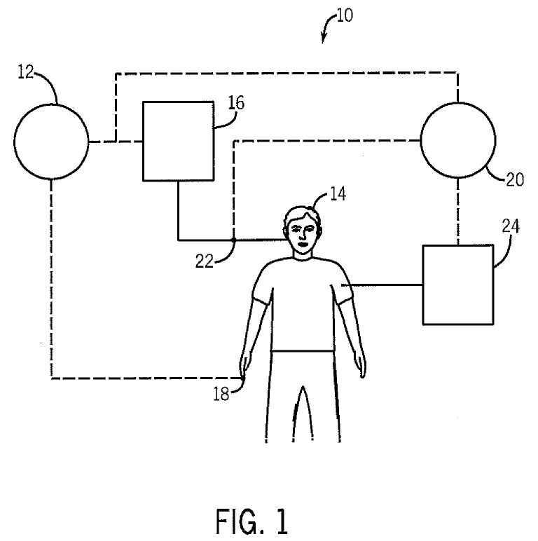

As indicated above, control of SpO₂ with oxygen delivery is presented herein as an example of a primary control feature in accordance with present embodiments. Specifically, FIG. 1 represents a system 10 including a primary controller 12 that controls a composition and/or delivery amount of a gas mixture to a patient 14 to safely induce, maintain, and/or control the patient's SpO₂ level. Thus, in an example embodiment, a gas mixture supplied to the patient 14 may be considered the delivery parameter. For example, automatic adjustment of FiO₂ by the primary controller 12 (e.g., a computer-based controller) may be utilized to control patient hypoxia or normoxia. FiO₂ may be defined as fractional inspired oxygen concentration or the percentage of oxygen in air inhaled by a patient through a ventilator. For example, in typical room air, the value for FiO₂ is approximately 21%. It should be noted that, in some embodiments, the primary controller 12 may account for circulatory delays based on a sensor location (e.g., an SpO₂ sensor located on a patient's finger tip) as discussed in U.S. patent application Ser. No. 11/524,182, which is incorporated herein by reference in its entirety

The primary controller 12 may include a closed-loop FiO₂ controller that cooperates with a ventilator 16 to control the patient's SpO₂ value. Indeed, the primary controller 12 may receive input from a sensor 18 (e.g., a pulse oximeter sensor) that measures the patient's SpO₂ value and, based on a comparison of the measured SpO₂ value with a target SpO₂ value, manipulate the ventilator's output (e.g., FiO₂). For example, the FiO₂ controller may output a request for increased FiO₂ from the ventilator 16 when a measured SpO₂ value is below a predefined SpO₂ target, or output a request for decreased FiO₂ from the ventilator 16 when the measured SpO₂ value is above the SpO₂ target. By increasing or decreasing FiO₂, the patient's lungs may receive more or less oxygen, respectively, and the value of SpO₂ in the patient will typically change correspondingly.

A secondary controller 20, such as a closed-loop treatment controller, in accordance with present embodiments, may utilize the output of the device (e.g., the ventilator 16) under the control of the primary controller 12 to regulate a separate medical therapy, identify certain patterns, and/or provide an alert based on the detection of certain conditions. For example, the secondary controller 20 may couple with a sensor 22 to monitor the actual FiO₂ provided through the ventilator 16 in response to the primary controller's request, and control certain procedures or alarm functions based on the measured value of the actual FiO₂. If the FiO₂ meets a predefined requirement, the secondary controller 20 may initiate an alarm or a procedure. For example, if the FiO₂ level exceeds a predefined value or has a specified correlation with respect to another value (e.g., the measured SpO₂), the secondary controller 20 may output a request for a device 24 to perform a particular operation (e.g., provide medication to the patient). In some embodiments, the secondary controller 20 may monitor the primary controller's request for FiO₂. In yet other embodiments, the secondary controller 20 may utilize both the request by the primary controller 12 and the actual measured FiO₂ level to determine an operation or output of the secondary controller 20.

By utilizing output generated by a medical device under closed-loop physiologic control, present embodiments may identify symptoms, additional physiologic parameters (e.g., lung diffusion capacity), provide alerts, and/or provide automated therapy or treatment. For example, with regard to the operation of the system 10 described above, the SpO₂ targeted FiO₂ may become elevated beyond a designated level, which may be indicative of certain patient issues. Such patient issues may be identified based on the traditional reaction of a patient to the delivery parameter (e.g., FiO₂) and/or correlations that typically exist between the delivery parameter and other parameters (e.g., SpO₂). For example, human lungs generally function to exchange oxygen and carbon dioxide between the body and the environment. Specifically, the lungs oxygenate blood, which eventually carries the oxygen throughout the body. Accordingly, changes in FiO₂ should directly affect the oxygenation of blood in the lungs over several breaths and blood in other areas of the body shortly thereafter. Thus, if the FiO₂ level, which is being supplied to the patient based at least in part on the patient's measured SpO₂ level, exceeds a predefined value, it may be indicative of reduced lung diffusion, reduced spontaneous breathing effort, and/or other issues relating to the oxygen diffusion capacity of the patient's lungs. Similarly, a ratio, difference or other correlation between the FiO₂ level and the measured SpO₂ level may be indicative of issues relating to the patient's health.

Accordingly, upon detecting that a delivery parameter generated by the primary controller 12 is at a certain level or upon detecting a certain correlation between the delivery parameter and a monitored physiological parameter, the secondary controller 20 may alert a clinician to perform an intervention and/or initiate automated treatment. For example, in conjunction with issuing an alarm notification, the secondary controller 20 may deliver pharmaceuticals (e.g., inhaled or injected pharmaceuticals), initiate suctioning of lung secretions, and/or inhibit patient-controlled analgesia. Several different medical interventions or therapeutic actions, such as those set forth above may be initiated by the secondary controller 20 depending on the primary controller's output and/or an algorithm of the secondary controller 20. For example, in the example discussed above, if the FiO₂ level meets a first requirement (e.g., a predefined threshold), the secondary controller 20 may initiate an alarm; if the FiO₂ level meets the first requirement and a second requirement, the secondary controller 20 may deliver pharmaceuticals to the patient 14; and if the FiO₂ level meets the first requirement, the second requirement, and a third requirement, the secondary controller 20 may initiate suctioning of the patient's lungs. In other embodiments, the secondary controller 20 may be capable of initiating separate actions based on a variety of different requirements or criteria. Further, different automated treatments and/or actions may be performed by the secondary controller 20 based on the output request of the primary controller 12.

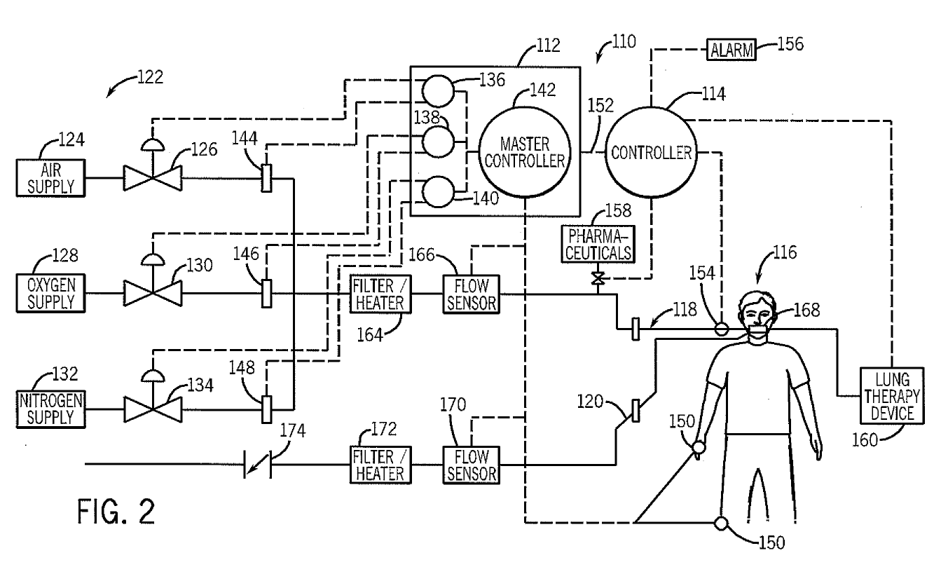

FIG. 2 is a block diagram of a medical system 110 in accordance with embodiments of the present disclosure. The medical system 110 may include a primary controller 112 for maintaining or controlling a physiologic parameter, and a secondary controller 114 for controlling therapy and/or providing an indication based at least in part on a delivery parameter or output generated by the primary controller 112. Specifically, the medical system 110 is represented as including a ventilation system with a controllable gas mixture supply mechanism, various therapeutic devices, an alert mechanism, the primary controller 112, and the secondary controller 114. Specific components and functions of the medical system 110 will be discussed in further detail below. It should be noted that while the embodiment illustrated by FIG. 2 is generally directed to patient ventilation, other embodiments may include features relating to other parameters and/or therapies.

The medical system 110 includes a ventilation feature that provides a patient 116 with a breathable gas mixture and receives the gas expelled by the patient 116. Specifically, the medical system 110 includes an inspiration line 118 and an expiration line 120. The inspiration line 118 facilitates provision of a controlled gas mixture for the patient 116 to breathe. The expiration line 120 receives gases (e.g., oxygen and carbon dioxide) exhaled by the patient 116. It should be noted that in some embodiments the medical system 110 includes an open exhalation line rather than the expiration line 120. In embodiments that implement the open exhalation line, gases exhaled by the patient do not pass back through the medical system 110 but simply pass directly into the atmosphere. Depending on application requirements, the open exhalation line or the expiration line 120 may be utilized to provide for safe operation or to facilitate certain procedures.

A ventilation inlet portion 122 of the medical system 110 includes an air supply 124 coupled to an air valve 126, an oxygen supply 128 coupled to an oxygen valve 130, and a nitrogen supply 132 coupled to a nitrogen valve 134. The inlet portion 122 may be capable of providing a defined gas mixture to the inspiration line 118. The supplies 124, 128, and 132 and valves 126, 130, and 134 may be utilized to produce normal, hyperoxic, and hypoxic gas mixtures for supply to the patient 116. Inclusion of the oxygen supply 128 may be desirable in some situations wherein a rapid increase in FiO₂ levels is desirable. However, it should be noted that some embodiments may not require hypoxic gas mixtures, and, thus, may not utilize the nitrogen supply 132.

Each of the gas supplies 124, 128, and 132 may include a high pressure tank or cylinder with pressurized air, nitrogen, or oxygen disposed respectively therein. The valves 126, 130, and 134 and/or additional valves may operate to normalize the pressure and ensure desired gas mixture proportions. In one embodiment, the air supply 124 may include the local atmosphere. That is, the air may be taken directly from the atmosphere using, for example, an air pump coupled to the air valve 126 in the ventilation inlet portion 122 of the medical system 110. Additionally, in some embodiments, a premixed gas supply may be provided and regulated with a gas mixture valve that facilitates combination with air or oxygen. The premixed gas may be supplemented with oxygen, air or both, and it may eliminate the use of the nitrogen supply 132.

Each of the valves 126, 130, and 134 in the ventilation inlet portion 122 of the medical system 110 may be a control valve, such as an electronic, pneumatic, or hydraulic control valve, that is communicatively coupled to a controller (e.g., flow controller or differential pressure controller), as illustrated by controllers 136, 138, and 140, respectively. The controllers 136, 138, and 140 may receive a set point value from a master controller 142 that controls SpO₂ levels in the patient 116. For example, each of the set points for the controllers 136, 138, and 140 may include a flow rate for each particular type of gas (e.g., air, oxygen, and nitrogen). To maintain or achieve a target SpO₂ level in the patient 116, the master controller 142 may supply set points or predefined curves (e.g., hysteresis curves) to the controllers 136, 138, and 140 such that levels of FiO₂ gradually fall or rise from a starting gas supply composition based on whether the patient needs more or less oxygen to reach the target SpO₂ level. The controllers 136, 138, and 140 may monitor flow sensors 144, 146, and 148 and open or close the valves 126, 130, and 134 depending on the amount of flow of each type of gas. These adjustments may maintain or control gas compositions in the inspiration line 118, as designated by the set points and/or curves from the master controller 142.

The illustrated controllers 136, 138, 140, and 142 may each include an input circuit configured to receive real-world data (e.g., a monitored physiological parameter of a patient) or other data (e.g., a set point from another controller). Additionally, the controllers 136, 138, 140, and 142 may each include an output circuit configured to provide signals (e.g., set point data) to a separate device or controller. For example, the output circuit may provide signals to an actuator or a set point value to a subordinate controller. Further, each controller 136, 138, 140, and 142 may include a memory storing an algorithm configured to calculate adjustments for maintaining or controlling physiologic parameters of the patient 116. Such algorithms (e.g., P, PD, PI, and PID algorithms) may be utilized to safely and efficiently bring the patient's physiological parameters to a desired state.

The master controller 142 may be programmed to maintain or control SpO₂ levels in the patient 116 by providing the set points and/or curves to the controllers 136, 138, and 140 such that valves 126, 130, and 134 open or close to supply an appropriate gas mixture composition (e.g., increased FiO₂ to achieve a higher SpO₂). For example, the master controller 142 may have a steady or dynamic set point based on a physiological condition of the patient 116, as monitored by one or more sensors 150 that detect physiological conditions of the patient 116. For example, a set point of the master controller 142 may be a predefined SpO₂ level in the patient 116 or a continuously changing SpO₂ level. It should be noted that in some embodiments, a controller (e.g., the primary controller 112) may simply control the amount of gas supplied to the patient 116, rather than the composition, to control certain physiologic parameters.

The controllers 136, 138, 140, and 142 may cumulatively be referred to as the primary controller 112. In the illustrated embodiment, each of the controllers 136, 138, 140, and 142 essentially cooperate to control at least one physiological parameter of the patient. Specifically, the controllers 136, 138, 140, and 142 essentially act as a single control mechanism for controlling SpO₂ by providing certain FiO₂ levels. In some embodiments, the controllers 136, 138, 140, and 142 may be replaced by a single controller capable of managing SpO₂ and FiO₂ levels. It should be noted that in embodiments relating to other physiological parameters, the primary controller 112 may control different time-varying therapeutic settings and the like. For example, the primary controller 112 may control positive end-expiratory pressure (PEEP) based on patient oxygenation. In yet another example, the primary controller 112 may control ventilation rate, inspired pressure, tidal volume and/or minute volume in response to a measure of CO₂ produced by the patient 116.

The primary controller 112 may include a pulse oximeter used to derive SpO₂ levels, or the primary controller 112 may be coupled to a pulse oximeter. Accordingly, the sensors 150 may include a pulse oximeter sensor and/or heart rate sensor that couples to the patient 116 to detect and facilitate calculation of the patient's SpO₂ and/or pulse. A pulse oximeter and its associated sensors may be defined as a device that uses light to estimate oxygen saturation of pulsing arterial blood. For example, light from a light emitting diode (LED) on the pulse oximeter sensor may be emitted into the patient's finger under control of the pulse oximeter and the light may be detected with a photodetector on the opposite side of the patient's finger. Using data gained through detecting and measuring the light with the pulse oximeter sensor, a percentage of oxygen in the patient's blood and/or the patient's pulse rate may be determined by the pulse oximeter. It should be noted that values for oxygen saturation and pulse rate are generally dependent on the patient's blood flow, although other factors may affect readings.

In one embodiment, the primary controller 112 may include a closed-loop FiO₂ controller configured to increase or decrease FiO₂ by an amount based at least in part on a difference between a measured SpO₂ value and an SpO₂ target value. Specifically, to control the patient's SpO₂ level, the primary controller 112 may manipulate FiO₂ levels based on a comparison of one or more stored SpO₂ set points and/or curves with pulse oximetry measurements of the patient's SpO₂ level taken via the sensor 150. For example, if the patient's SpO₂ level is above a target level, the primary controller 112 may reduce FiO₂ by increasing the amount of nitrogen feed (e.g., increasing flow through the nitrogen valve 134) by increasing the corresponding controller set point) while decreasing oxygen levels (e.g., decreasing flow through the oxygen and/or air valves 126 and 130) by decreasing the corresponding controller set points) in the inspiration line 118. Additionally, the primary controller 112 may manipulate FiO₂ levels in response to heart and respiration rates that may also be monitored by the sensors 150, which may include respiration sensors. For example, if the patient's heart rate exceeds a set value or if the respiration rate exceeds a set value, the primary controller 112 may increase FiO₂ by increasing oxygen.

The secondary controller 114 may utilize output generated by the primary controller 112 to essentially diagnose and/or address a patient's situation. For example, the secondary controller 114 may receive a controller output 152 (e.g., an electronic or pneumatic signal) directly from the primary controller 112 and/or a value of a measured delivery parameter (e.g., a measured FiO₂ value) from a sensor 154. Because the primary controller 112 is capable of controlling a physiological parameter, the delivery parameter may provide additional information about the patient's status. Indeed, the secondary controller 114 may utilize algorithms stored within a memory or other tangible computer-readable medium to identify symptoms, identify additional physiologic parameters, provide alerts, and/or provide automated therapy or treatment.

Specifically, for example, upon detecting that an FiO₂ level, which is being supplied to the patient based at least in part on the patient's measured SpO₂ level, exceeds a predefined value, the controller 114 may identify a pattern or threshold corresponding to reduced lung diffusion, reduced spontaneous breathing effort, lung edema, atalectasis, pulmonary embolism, and/or other issues relating to the oxygen diffusion capacity of the patient's lungs. Accordingly, the secondary controller 114 may activate an alarm 156, initiate supply of pharmaceuticals (e.g., release inhaled pharmaceuticals into the inspiration line 118 or inject pharmaceuticals into the patient 116) from a pharmaceutical supply mechanism 158, activate a lung therapy device 160, and/or activate various other therapies or alerts. It should be noted that the alarm 156 may include a device capable of providing a visual, haptic, and/or audible indicator. For example, the alarm 156 may include a display screen and speakers capable of describing a condition.

In some embodiments, different therapies and/or alarms may be activated depending on a ratio, correlation, and/or other relationship relative to the delivery parameter. Indeed, in some embodiments, a sequence of different actions may be taken as patterns relating to the delivery parameter change. For example, if the secondary controller 114 detects that a patient's status is becoming more critical based on changes in the delivery parameter and/or other data (e.g., the physiological parameter driving the delivery parameter), the actions taken by the secondary controller 114 may become more aggressive. Indeed, as a patient's condition steadily becomes more critical, the secondary controller 114 may increase the intensity of a warning issued by the alarm 156 and/or increase the nature of therapeutic intervention. For example, the secondary controller 114 may initiate introduction of pharmaceuticals from the pharmaceutical supply mechanism 158 based on a recognized pattern relating to the delivery parameter. If the pattern continues or changes to suggest a more critical condition in the patient 116, the controller 114 may provide additional pharmaceuticals or initiate a different therapy, such as suctioning the patient's lungs with the lung therapy device 160.

In operation, systems in accordance with present embodiments, such as the system 110 illustrated in FIG. 2, generally function to control a physiological parameter with a delivery parameter. Further, based on the delivery parameter, present embodiments may identify and/or treat additional physiological parameters, patient conditions, and the like. While various different physiological parameters, delivery parameters, control algorithms, and so forth may be utilized, the general operation of the system 110 may be exemplary of present embodiments. For example, it is believed that the primary controller 112 of the system 110, which may include an SpO₂-targeted FiO₂ control algorithm, will maintain the patient's SpO₂ relatively constant. Accordingly, changes in the FiO₂ level may be indicative of changes in the oxygen diffusion capacity of the patient's lungs. Thus, the primary controller's closed-loop control based on the targeted physiological parameter essentially transforms the FiO₂ level, which has traditionally been a therapeutic setting that was left unchanged for hours or days, into a real-time indication of a different, and possibly more specific physiologic parameter (e.g., lung diffusion capacity) that may not otherwise be readily available.

Specifically, during operation of the system 110, the primary controller 112 receives an SpO2 value for the patient 116 and provides a corresponding FiO2 level to facilitate achieving or maintaining a desired SpO2 value. In the illustrated embodiment, this may be achieved by an algorithm in the master controller 142 that provides set points to the subordinate controllers 136, 138, and 140. After being mixed or flowed according to the set points determined by master controller 142, a gas mixture may proceed from the inlet ventilation portion 122 of the system 110 along the inspiration line 118 to a filter/heater 164. The filter/heater 164 may operate to filter out bacteria, remove other potentially harmful or undesirable elements, and heat the gas mixture to a desired temperature. Upon exiting the filter/heater 164, the gas mixture may proceed to a flow sensor 166 (e.g., a differential pressure sensor) that measures a total flow rate of the gas mixture to the patient 116 through the inspiration line 118. Values obtained from the flow sensor 166 may be utilized in control and maintenance of patient SpO₂ levels by providing information for use in algorithms of the primary controller 112 and/or secondary controller 114. Eventually, the gas mixture may exit the system 110 and may be delivered to the patient 116 via a delivery piece 168 (e.g., endotracheal tube, laryngeal mask airway, face mask, nasal pillow, or nasal canula).

Several implementations of the expiration line 120 may be utilized to handle gases (e.g., CO₂ and O₂) exhaled by the patient 116. For example, different exhalation sensors, filters, heaters, and configurations may be utilized dependent upon the patient's needs and/or other desirable conditions. In the embodiment illustrate by FIG. 2, gases exhaled by the patient 116 may be received back into the system 10 via the expiration line 120. Once received, the exhaled gases may proceed through a flow sensor 170, which measures values associated with the exhaled gases (e.g., a volumetric flow rate). Information from the flow sensor 170 may be utilized by the primary controller 112 and/or the secondary controller 114 to further adjust parameters that relate to safely maintaining patient physiological parameters, diagnosing patient conditions, and/or providing therapy. For example, flow rates of exhaled air from the patient may be utilized in an algorithm of the primary controller 112 and/or the secondary controller 114 to compare with a predefined minimum exhalation rate for the patient. A difference between flow sensors 166 and 170 may be used to determine when the patient 116 inspires or exhales, the patient's inspired or exhaled flow may then be integrated to determine inspired or exhaled volume. Respiratory parameters such as respiratory rate and minute volume may then be calculated and used as auxiliary inputs to the primary controller 112 and/or secondary controller 114.

Upon exiting the flow sensor 170, the exhaled gas may proceed to a filter/heater 172, to a check valve 174, and out of the system 110. The filter/heater 172 may be adapted to cleanse the exhaled gases, and the check valve 174 may operate to prevent the exhaled gases from circulating back to the patient 116 through the system 110.

The delivery parameter resulting from the primary controller's closed-loop control of SpO₂, as measured by the sensor 154 and/or determined by the direct output from the primary controller 112, may be received by the secondary controller 114. Based on this delivery parameter and/or other available data (e.g., the difference in inhaled and exhaled flow rates and/or SpO₂), the secondary controller 114 may utilize an algorithm stored on a computer-readable medium to analyze data patterns with respect to the delivery parameter and/or the physiologic parameter being controlled by the primary controller 112. Based on this analysis, the secondary controller 114 may initiate and/or adjust various therapies and alarms. In some embodiments, this may include various levels of response and/or adjustment. For example, as indicated above, the primary controller 112 may adjust an alarm level being emitted by the alarm 156, adjust a flow rate of pharmaceuticals from the pharmaceutical supply mechanism 158, and/or activate the lung therapy device 160. A user (e.g., a clinician) may designate the order and/or level of responses based on particular patients. In some embodiments, certain treatment scenarios may be programmed into an algorithm based on certain patient traits, such as a particular disease state or medical history.

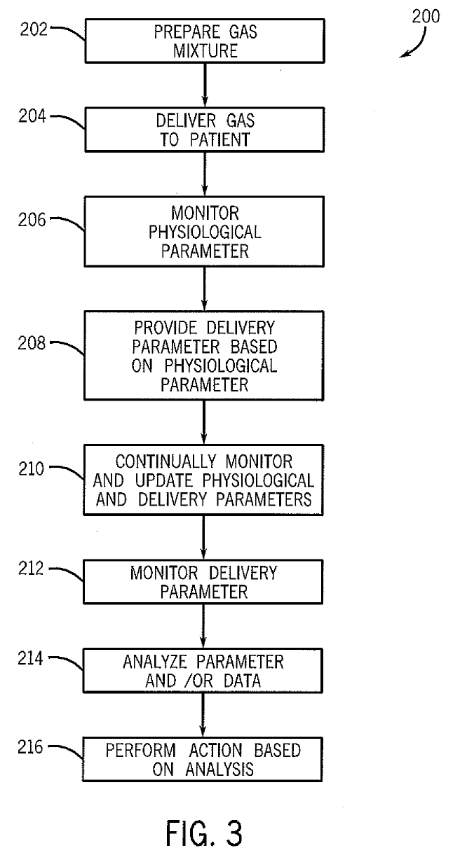

FIG. 3 is a block diagram of a method illustrating an exemplary embodiment of the present invention. The method is generally referred to by reference number 200. The method 200 may be implemented by a computer (e.g., a computer-based controller), based on an algorithm stored on a tangible, computer-readable medium (e.g., a memory component of a controller). Specifically, the method 200 begins with preparation of a breathable gas mixture (block 202). For example, block 202 may include mixing gases from the supplies 124, 128, and 132 in the inlet portion 122 of the system 110 to maintain a certain FiO₂ using the primary controller 112, and valves 126, 130, and 134 based on data received from sensors (e.g., sensors 150, 166, and 170). In other embodiments, block 202 may include manipulating an amount of gas (e.g., reducing or increasing flow) provided to a patient to maintain a certain FiO₂. Next, block 204 represents delivering the gas mixture to a patient, as may be achieved via the inspiration line 118 of the system 110 illustrated by FIG. 2.

Block 206 represents monitoring at least one physiological parameter (e.g., SpO₂) of the patient. Block 208 represents providing at least one delivery parameter (e.g., FiO₂) to the patient to adjust or maintain the physiological parameter monitored in block 206. By continually monitoring patient physiological parameters and updating delivery parameters or output, as illustrated by block 210, embodiments of the present disclosure may maintain or control certain physiologic parameters of a patient, such as a patient's SpO₂ level. For example, this can be achieved using the primary controller 112 of the system 110.

Block 212 represents monitoring the delivery parameter or an output from control of the physiological parameter. Specifically, block 212 may represent monitoring the delivery parameter provided in block 208. For example, the secondary controller 114 may monitor the delivery parameter generated based on operation of the primary controller 112. Block 214 represents analyzing the delivery parameter and/or other data (e.g., physiological parameters) to identify a pattern or scenario relating to a patient's health. For example, the secondary controller 114 may utilize an algorithm to determine that the FiO₂ level is indicative of a particular condition in view of the patient's SpO₂ level. The algorithm may take into account historic data (e.g SpO₂ or FiO₂ trends, or an X-ray image of the lungs), a patient's historic medical information (e.g., an existing heart condition), a user specified protocol, a protocol designated for certain types of patients, and the like. Block 216 represents performing an action based on the analysis performed in block 214. For example, block 216 may represent supplying the patient with a pharmaceutical or initiating a therapeutic action. The action taken based on the analysis of block 214 may be automatic intervention or the action may include notifying a clinician that intervention may be desirable.

In some embodiments, other procedures and/or different procedures may be implemented to facilitate, improve, or achieve diagnostic and/or therapeutic results. Indeed, embodiments of the present disclosure are applicable to systems with time-varying therapeutic settings other than FiO₂ and having targeted physiological parameters other than SpO₂. For example, a system that adjusts PEEP based on a measure of patient oxygenation may also automatically inhibit or reduce patient-controlled analgesia, deliver a pharmaceutical (e.g., a bronchodilator), and/or initiate suctioning. Such actions may be taken when a requested PEEP level is adjusted beyond some predetermined level or beyond some predetermined duration. Further, a system that automatically adjusts settings such as ventilation rate, inspired pressure, tidal volume, or minute volume in response to a measure of CO₂ might deliver or adjust therapy. For example, in response to levels of the automatically adjusted settings in excess of a threshold and depending on the likely cause of the levels of the automatically adjusted settings, the system may act to slow metabolism, reduce sedation, improve circulation, improve lung compliance and/or improve lung resistance. Conversely, in response to low levels of the automatically adjusted settings, such a system may deliver or adjust a therapy to increase metabolism. Similarly, a system that automatically adjusts a therapy, such as provision of epinephrine to maintain a targeted level of cardiac output or blood pressure, may also signal a ventilator to decrease PEEP if the therapy had to be adjusted above a certain level. A system that automatically adjusts an antiarrhythmic therapy based on cardiac rhythm may signal the ventilator to increase oxygen delivery or increase an SpO₂ target for control of oxygen delivery, if the level of antiarrhythmic therapy had to be increased beyond some predetermined limit. A system that controls the delivery of inhaled or intravenous anesthetics in order to target a specified depth-of-anesthesia might, if the required anesthesia level got too high, also direct a surgical cutting device to inject local anesthetic as it cuts. A system that controls fluid delivery to a tissue-hydration target might also adjust diuretic levels if the fluid delivery rate exceeded preset limits. A system that controls diuretic levels so as to achieve a targeted urine production might also adjust fluid delivery rates if the diuretic level had to be adjusted above some predetermined level.

It should be noted that the types of intervention or therapy set forth in the present disclosure may require knowledge of a patient's diagnosis and/or history. Accordingly, a qualified user (e.g., a physician) may supply appropriate thresholds, exclusion criteria, response times, correlations, and so forth for algorithms employed by controllers in accordance with present embodiments. Such settings may be based on institutional protocols in conjunction with diagnostic information that may be input by a user or automatically extracted from storage (e.g., an electronic patient record). Where multiple interventions are enabled, certain interventions may be prioritized. Further, other operational factors may be designated, such as a number to times to repeat a particular intervention based on effects.

While the invention may be susceptible to various modifications and alternative forms, specific embodiments have been shown by way of example in the drawings and will be described in detail herein. However, it should be understood that the invention is not intended to be limited to the particular forms disclosed. Rather, the invention is to cover all modifications, equivalents and alternatives falling within the spirit and scope of the invention as defined by the following appended claims.

What is claimed is:

1. A method for automated medical treatment, comprising:
monitoring a physiological parameter of a patient with a first control feature;
supplying the patient with a delivery parameter based at least in part on the physiological parameter to control the physiological parameter;
determining a therapeutic procedure to be performed on the patient with a second control feature based at least in part on analysis of the delivery parameter; and
automatically initiating an alert indicative of the therapeutic procedure and/or automatically performing the therapeutic procedure.

2. The method of claim 1, wherein monitoring the physiological parameter comprises monitoring the patient's blood oxygen saturation.

3. The method of claim 1, wherein supplying the patient with the delivery parameter comprises supplying the patient with a determined level of oxygen.

4. The method of claim 1, comprising automatically supplying the patient with a pharmaceutical.

5. The method of claim 1, comprising automatically suctioning lung secretions from the patient.

6. The method of claim 1, comprising displaying a description of the therapeutic procedure.

7. The method of claim 1, wherein determining the therapeutic procedure comprises analyzing the patient's medical history and/or designated protocols.

8. The method of claim 1, comprising receiving diagnostic input to facilitate determining the therapeutic procedure.

9. The method of claim 1, comprising automatically performing a plurality of therapeutic procedures based at least in part on the analysis of the delivery parameter and a prioritization of the plurality of therapeutic procedures.

10. A medical system, comprising:
a primary controller capable of monitoring a physiological parameter of a patient and capable of controlling the physiological parameter by adjusting a delivery parameter; and
a secondary controller capable of monitoring the delivery parameter and automatically performing a therapeutic procedure based at least in part on the delivery parameter.

11. The medical system of claim 10, wherein the secondary controller is capable of determining the therapeutic procedure to be performed on the patient based at least in part on analysis of the delivery parameter.

12. The medical system of claim 10, wherein the secondary controller is capable of receiving a diagnostic protocol and/or medical history data and analyzing the delivery parameter based at least in part on the diagnostic protocol and/or the medical history data.

13. The medical system of claim 10, wherein the secondary controller is capable of activating a therapeutic device.

14. The medical system of claim 13, wherein the therapeutic device comprises a pharmaceutical supply device.

15. The medical system of claim 13, wherein the therapeutic device comprises a bodily fluid removal device.

16. The medical system of claim 10, comprising a plurality of controllers capable of activating a plurality of therapeutic devices.

18. The medical system of claim 10, comprising a pulse oximeter.

19. A medical system, comprising:
a pulse oximeter monitor capable of controlling a blood oxygen saturation level of a patient by detecting the patient's blood oxygen saturation and supplying a gas mixture to the patient based at least in part on the patient's blood oxygen saturation and a target blood oxygen saturation level; and
a treatment controller capable of controlling a therapeutic procedure by monitoring an output of the pulse oximeter monitor and/or the gas mixture and controlling the therapeutic procedure based at least in part on the output and/or the gas mixture.

20. The medical system of claim 19, wherein the therapeutic procedure comprises injecting the patient with a pharmaceutical using a pharmaceutical supply mechanism and/or removing fluid from the patient using a lung therapy device.
